(19) United States
(12) Patent Application Publication
(10) Pub. No.: US 2015/0366405 A1
(43) Pub. Date: Dec. 24, 2015

(54) METHOD AND APPARATUS FOR MAKING CUSTOMIZED NUTRITIONAL MIXTURES

(71) Applicant: Aleh Manchuliantsau, Vitebsk (BY)

(72) Inventor: Aleh Manchuliantsau, Vitebsk (BY)

(21) Appl. No.: 14/745,674

(22) Filed: Jun. 22, 2015

Related U.S. Application Data
(60) Provisional application No. 62/015,386, filed on Jun. 20, 2014.

(51) Int. Cl. A47J 43/07; A61B 5/00; A61B 5/145; A61B 5/0205

(52) U.S. Cl. CPC A47J 43/07; A61B 5/0205; A61B 5/4504; A61B 5/4866; A61B 5/14542

(57) ABSTRACT

This disclosure relates to a method and an apparatus for making customized nutritional mixtures based on characteristics of an individual and in the manner to be nutritionally complete and correspond to a recommended calorie intake value. An example method comprises the steps of: (i) maintaining a plurality of nutritional ingredients; (ii) receiving a request to make a customized nutritional mixture for an individual; (iii) obtaining or calculating an individual calorie value of the individual; (iv) optionally selecting a number of nutritional ingredients; (v) calculating mass fractions of selected nutritional ingredients based on the individual calorie value; and (vi) dispensing selected nutritional ingredients in accordance with the mass fractions to a container in order to produce the customized nutritional mixture, where a calorie value of the customized nutritional mixture substantially corresponds to at least a predetermined portion of the individual calorie value.

### CROSS-REFERENCE TO RELATED APPLICATIONS

This application claims the benefit of U.S. provisional patent application Ser. No. 62/015,386 filed on Jun. 20, 2014, entitled “Method and Apparatus for Making Customized Nutritional Mixtures,” which is incorporated herein by reference in its entirety for all purposes.

### TECHNICAL FIELD

This disclosure generally relates to methods and systems for making nutritional mixtures. More specifically, this disclosure relates to a method and apparatus for making customized nutritional mixtures based on characteristics of an individual and in the manner to be nutritionally complete and correspond to a recommended calorie intake value as described and shown herein.

### BACKGROUND

The approaches described in this section could be pursued, but are not necessarily approaches that have been previously conceived or pursued. Therefore, unless otherwise indicated herein, the approaches described in this section are not prior art to the claims in this application and are not admitted to be prior art by inclusion in this section.

Today, vending machines, automated cooking machines, and automated food dispensers are widely known in the art. Vending machines typically provide dispensing of items, such as pre-packaged foodstuff and beverages to customers, in response to acceptance of the customer's payment. Cooking machines and cooking appliances provide preparing foodstuff or beverages based on one or more ingredients stored inside the machine. There are also known cooking vending machines, which are a combination of a traditional vending machine and a cooking machine. The cooking vending machine can typically prepare foodstuff or beverages, such as coffee or hot chocolate, by dispensing two or more components from storage containers in measured amounts into a single receptacle (e.g., a cup, which the customer removes from the machine when the beverage is ready).

The traditional machines are known to dispense or cook foodstuff based on a selection of customer. Typically, a customer inputs a serving choice or selects a pre-packaged product, and in response to the customer input, the machine provides foodstuff or beverage. In some machines, pre-stored programs enable creating customized foodstuffs or drinks based on user selection and limited rules governing the dispensing of the ingredients.

### SUMMARY

This summary is provided to introduce a selection of concepts in a simplified form that are further described below in the Detailed Description section. This summary is not intended to identify key features or essential features of the claimed subject matter, nor is it intended to be used as an aid in determining the scope of the claimed subject matter.

The present technology relates to a method and apparatus for making customized nutritional mixtures, such as dry mixes, smoothies or drinks, for an individual based at least in part on a daily intake calorie value and nutritional values recommended for the individual. The making of customized nutritional mixtures can also depend on, but not limited to, goals of the individual, selections or preferences of the individual, biophysical parameters of the individual, health information of the individual, vital parameters of the individual, physical activity information of the individual, medical information of the individual, and/or predetermined nutrition selection rules. Accordingly, the present technology allows for making nutritionally complete mixtures, which correspond to recommended target calorie intake values individually calculated based on multiple factors and individual goals. The mixtures are made to contain substantially any and all natural ingredients sufficient to meet nutritional requirements of a predetermined calorie diet, such as U.S. Department of Agriculture (USDA) 2,000 calories diet. Thus, the custom-tailored mixtures according to this disclosure allow the individuals to remain healthy, build muscles, and lose weight depending on particular needs and individual characteristics. The technology also allows for packaging the mixtures in containers or bottles for further convenient use.

According to one aspect of this disclosure, there is provided a method for making customized nutritional mixtures. The method comprises the steps of: (i) maintaining a plurality of nutritional ingredients; (ii) receiving, by means of a control module comprising a processor and a memory storing program instructions executable by the processor, a request to make a customized nutritional mixture, wherein the request is associated with an individual; (iii) obtaining, by means of the control module, an individual calorie value associated with the individual; (iv) calculating, by means of the control module, mass fractions of nutritional ingredients based on the individual calorie value; and (v) dispensing, by means of a dispensing module, selected nutritional ingredients in accordance with the mass fractions to a container in order to produce the customized nutritional mixture, wherein a calorie value of the customized nutritional mixture substantially corresponds to a predetermined portion of the individual calorie value.

According to some embodiments of this disclosure, the method may further comprise the step of selecting, by means of the control module, a number of nutritional ingredients of the plurality of nutritional ingredients based on one or more predetermined rules and individual preferences. According to more embodiments of this disclosure, the method may further comprise the step of dispensing, by means of a secondary dispensing module, a consumable liquid to the container. According to some additional embodiments of this disclosure, the method may further comprise the step of mixing, by means of a mixing module, the selected nutritional ingredients inside the container.

In certain embodiments, the individual calorie value can characterize a recommended calorie target value for the individual to consume within a predetermined period, wherein the predetermined period is between 1 hour and 24 hours. The request of the individual to make the customized nutritional mixture can be received from a customer interface based on an input of the individual. In some embodiments, the individual calorie value is included in the request of the individual to make the customized nutritional mixture, and the obtaining of the individual calorie value includes retrieving the individual calorie value from the request. In other embodiments, the step of obtaining of the individual calorie value comprises receiving, by means of the control module, the individual calorie value calorie value from a mobile device associated with the individual.

In certain embodiments, the step of obtaining of the individual calorie value comprises receiving, by means of the control module, the individual calorie value from a wearable computing device associated with the individual, wherein the wearable computing device includes at least one sensor for sensing one or more vital parameters of the individual. In some embodiments, the step of obtaining of the individual calorie value comprises receiving, by means of the control module, the individual calorie value from a website, a web service, and/or a web resource.

According to some additional embodiments of this disclosure, the method may further comprise the step of receiving, by means of the control module, biophysical parameters of the individual, wherein the obtaining of the individual calorie value comprises calculating the individual calorie value based upon the biophysical parameters of the individual. In certain embodiments, the biophysical parameters of the individual comprise a gender of the individual, an age of the individual, a height of the individual, a weight of the individual, and a daily activity level of the individual. In certain embodiments, the biophysical parameters of the individual comprise one or more of the following vital parameters: a blood pressure of the individual, a heart rate of the individual, a blood oxygen level of the individual, a value of calories consumed by the individual within a predetermined period, and a bone thickness of the individual. In certain embodiments, the biophysical parameters of the individual comprise one or more of the following physical activity parameters: a number of steps made by the individual for a period of time, a distance travelled by the individual, sleep quality parameters of the individual, a number of calories burned off by the individual, and a type of physical activity of the individual.

In certain embodiments, the step of calculating the calorie value is based on a Body Mass Index (BMI) associated with the individual. In other embodiments, the step of calculating the calorie value is based on a Basal Metabolic Rate (BMR) or a Resting Metabolic Rate (RMR) associated with the individual. In certain embodiments, the step of calculating of the mass fractions of the nutritional ingredients is further based on one or more nutritional ingredient selection rules associated with a calorie diet, wherein the calorie diet is associated with the individual and based on 1,000 to 4,000 calorie daily intake.

In certain embodiments, the predetermined rules include one or more ratios of the nutritional ingredients associated with multiple calorie values. According to some additional embodiments of this disclosure, the method may further comprise the step of mapping, by means of the control module, the one or more ratios of the nutritional ingredients to the individual calorie value associated the individual. In certain embodiments, the one or more ratios of the nutritional ingredients comprise first ratios associated with calorie sources and second ratios associated with minerals and vitamins, wherein the calorie sources include fats, carbohydrates, and proteins. In certain embodiments, the vitamins comprise: Vitamin A, Vitamin B6, Vitamin B12, Vitamin C, Vitamin D, Vitamin E, Vitamin K, thiamin, riboflavin, niacin, folate, and choline. In certain embodiments, the minerals comprise calcium, copper, iron, magnesium, phosphorous, selenium, zinc, potassium, and sodium.

In certain embodiments, the step of calculating of the mass fractions of the selected nutritional ingredients is performed such that the selected nutritional ingredients correspond to at least one of the first ratios and at least one of the second ratio. In certain embodiments, the first ratios include: 10%-40% of fats. 40%-80% of carbohydrates, and 10%-50% of proteins. In certain embodiments, the first ratios include: about 30% of fats, about 60% of carbohydrates, and about 10% of proteins.

According to some additional embodiments of this disclosure, the method may further comprise the step of selecting, by means of the control module, one of the ratio of the nutritional ingredients based on a gender of the individual, an age of the individual, a height of the individual, a weight of the individual, and a daily activity level of the individual. According to some additional embodiments of this disclosure, the method may further comprise the step of selecting, by means of the control module, one of the ratio of the nutritional ingredients based on one or more of the biophysical parameters associated with the individual.

In certain embodiments, the nutritional ingredients include: flax seeds and hulled hemp seeds. In certain embodiments, the nutritional ingredients include: flour from at least one gluten-free plant, flax seeds, hulled hemp seeds, dextrose, and protein. In certain embodiments, the predetermined portion of the individual calorie value is between 10% and 50% of the individual calorie value, wherein the individual daily calorie value is between 100 calories and 4,000 calories. In other embodiments, the predetermined portion of the individual daily calorie value is at least 100% or more of the individual daily calorie value, wherein the individual daily calorie value is between 100 calories and 4,000 calories.

According to another aspect of this disclosure, there is provided an apparatus for making customized nutritional mixtures. The apparatus comprises: (i) a plurality of ingredient storage modules for storing a plurality of nutritional ingredients; (ii) a dispensing module for dispensing nutritional ingredients to a container; (iii) a control module comprising a processor and a memory storing program instructions executable by the processor, the control module is operatively coupled to the dispensing module. The control module is configured to: receive a request to make a customized nutritional mixture, wherein the request is associated with an individual; obtain an individual calorie value associated with an individual; calculate mass fractions of nutritional ingredients based on the individual calorie value; and cause the dispensing module to dispense selected nutritional ingredients in accordance with the mass fractions to a container in order to produce the customized nutritional mixture, wherein a calorie value of the customized nutritional mixture substantially corresponds to at least a predetermined portion of the individual calorie value.

In certain embodiments, the apparatus may further comprise a customer interface for displaying information to the individual and for receiving the request of the individual. In certain embodiments, the apparatus may further comprise a communication module for receiving the request of the individual from a mobile device, a wearable computer, or a server.

According to another aspect of this disclosure, there is provided a non-transitory processor-readable medium having instructions stored thereon, which when executed by one or more processors, cause the one or more processors to implement a method for making customized nutritional mixtures, the method comprising: receiving a request to make a customized nutritional mixture, wherein the request is associated with an individual; obtaining an individual calorie value associated with an individual; calculating mass fractions of nutritional ingredients based on the individual calorie value; and causing to dispense selected nutritional ingredients in accordance with the mass fractions to a container in order to produce the customized nutritional mixture, wherein a calorie value of the customized nutritional mixture substantially corresponds

Additional objects, advantages, and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

Embodiments are illustrated by way of example and not limitation in the figures of the accompanying drawings, in which like references indicate similar elements and in which:

FIG. 1 illustrates an example apparatus for making customized nutritional mixtures.

FIG. 2 illustrates a high-level block diagram of example apparatus for making customized nutritional mixtures.

FIG. 3 illustrates a high-level block diagram of a system environment for operating an apparatus for making customized nutritional mixtures.

FIG. 4 illustrates a process flow diagram showing an example method for making customized nutritional mixtures.

### DETAILED DESCRIPTION

The following detailed description includes references to the accompanying drawings, which form a part of the detailed description. The drawings show illustrations in accordance with example embodiments. These example embodiments, which are also referred to herein as “examples,” are described in enough detail to enable those skilled in the art to practice the present subject matter. The embodiments can be combined, other embodiments can be utilized, or structural, logical and operational changes can be made without departing from the scope of what is claimed. The following detailed description is, therefore, not to be taken in a limiting sense, and the scope is defined by the appended claims and their equivalents.

As already outlined above, this disclosure relates to a method and apparatus for making customized nutritional mixtures such as dry mixes, blends, drinks, smoothies, beverages, or any other suitable form. The mixtures are nutritionally complete meal and correspond to predetermined and recommended calorie intake value individually calculated for each customer based on individual characteristics, individual goals, and other factors.

According to embodiments of this disclosure, the customized nutritional mixtures are prepared in amounts sufficient to intake on a daily basis or another period (e.g., per meal). There is no need for an individual to intake any other meals or foodstuff, because the customized nutritional mixtures are nutritionally complete food, which provides about 97%-100% of recommended amounts of nutrients, minerals, and vitamins for each individual. These recommended amounts are calculated based on individual characteristics (e.g., a gender, age, height, weight, activity level), individual goals (e.g., to remain healthy, to lose weight, to gain muscles), and nutritional rules (e.g., USDA standards). Optionally, the recommended amounts of nutrients, minerals, and vitamins are calculated based on biophysical parameters (e.g., blood pressure, heart rate, calories burned, distances traveled, etc.). Moreover, the customized nutritional mixtures correspond to recommended target calorie intake value (e.g., to meet 2,000 calories diet), recommended amounts of fat, carbs and protein.

Generally, customized nutritional mixtures can be prepared from multiple and various ingredients, and this disclosure is not limited to any particular recipes required to make nutritionally complete meals, but merely provides some example embodiments. Those skilled in the art will appreciate that the embodiments of this disclosure can be practiced to make various nutritional mixtures.

The term “nutritional mixture,” as used herein, shall mean any blend or mix prepared from two or more ingredients. The nutritional mixture can be dispensed or mixed, or can be further processed to take any suitable form including, but not limited to, a dry mixture, beverage, drink, smoothie, jelly, soup, snack bar, bakery product, and so forth. The ingredients may be of the dry form, solid form, liquid form, and/or semi-liquid form. The nutritional mixtures can be dispensed, mixed or packaged in a container.

The term “container,” as used herein, shall mean any compressible or non-compressible container or receptacle usually used for mixing and/or storing solids, liquids, or semi-liquids. Some examples of the container include, but not limited to, bottle, flask, package, packet, wrap, pot, beaker, vat, cask, barrel, pitcher, bowl, and cup. Containers, when filled with nutritional mixtures, can be used by individuals for further intake. In some embodiments, individuals just need to add water or other liquid to a container with the customized nutritional mixture, shale and then drink.

The term “individual,” as used herein, shall mean any person or animal that can consume customized nutritional mixtures. The term “individual” also mean a user, consumer, customer, patient, operator, and the like.

According to embodiments of this disclosure, there is provided an apparatus for making customized nutritional mixtures, which can include, but not limited to, a commercial vending machine, self-service trading machine, commercial cooking appliance, manufacturing equipment, and cooking appliance for use at home or office. The apparatus can also refer to a commercial kitchen appliance for use by catering companies. The apparatus can be implemented in the form of a single assembly or a multi-component system.

According to various embodiments of the present disclosure, the apparatus for making customized nutritional mixtures includes a plurality of ingredient storage modules for storing a plurality of nutritional ingredients, a dispensing module for dispensing nutritional ingredients to a container, and a control module. The control module may refer to a computing device and include at least a processor and a memory, which can store program instructions executable by the processor. The control module can operatively be coupled at least to the dispensing module. In some embodiments, the apparatus includes a customer interface for displaying information to an individual and receiving inputs of the individual. In yet other embodiments, the apparatus includes a communication module for receiving individual inputs, requests and selections from a remote device, such as a mobile device, wearable computer, server, website, web service, or web resource.

According to some embodiments, the control module may operate as follows. First, the control module can receive a request of an individual to make a customized nutritional mixture. The request may include biophysical parameters of the individual such as age, height, weight, gender, activity level. Optionally, the request may include individual goals (e.g., to remain healthy, to lose weight, to gain muscles). The request may also include vital parameters such as a blood pressure of the individual, a heart rate of the individual, a blood oxygen level of the individual, a value of calories consumed by the individual within a predetermined period, and a bone thickness of the individual. The request may also include physical activity parameters such as a number of steps made by the individual for a period of time, a distance travelled by the individual, sleep quality parameters of the individual, a number of calories burned off by the individual, and a type of physical activity of the individual. In yet more embodiments, the request may include, for example, a selection of serving choice, serving size, type of container used, nutritional mixture flavor, and so forth.

Further, the control module obtains a calorie value associated with the individual. The calorie value may refer to a recommended calorie intake value required for the individual to intake. For example, the recommended calorie intake value may be 2,000 calories per day, but not necessary. In various embodiments, the recommended calorie intake value may vary between 1,000 and 4,000 calories per day. Notably, the recommended calorie intake value can be input by the individual, or, alternatively, the calorie value can be calculated based on various biophysical parameters, vital and physical activity parameters associated with the individual. For example, the individual can provide answers to a questionnaire or input personal and/or health information, such that the control module can calculate a recommended calorie intake value required for the individual to intake. In other embodiments, the calorie value and/or biophysical information can be acquired from an individual's mobile device, mobile applications, wearable computing device, medical sensors, sport or physical training trackers, websites, web resources, web services, and so forth. In case the calorie value cannot be obtained, it would be calculated by the control module based on available biophysical information of the individual. In yet more embodiments, the apparatus for making customized nutritional mixtures can include one or more sensors configured to measure biophysical or other characteristics of an individual (e.g., weight, height, heart rate, blood pressure, etc.) which can be used in calculation of recommended calorie intake value.

Once the recommended calorie intake value is obtained, the control module selects a number of nutritional ingredients of the plurality of nutritional ingredients based on predetermined rules and optionally on individual characteristics and preferences. The control module also calculates mass fractions (e.g., dosages) for the selected nutritional ingredients based on the recommended calorie intake value and the predetermined rules. Further, the control module controls the dispensing module to dispense the selected nutritional ingredients in accordance with the calculated mass fractures to a container. In some embodiments, a mixer can be used to mix or stir the selected and dispensed nutritional ingredients to make a homogeneous substance. The mixing can occur in the container or in a mixer.

In certain embodiments, the apparatus for making customized nutritional mixtures may include additional modules. For example, the apparatus may include a payment module configured to acquire monetary or non-monetary payments from individuals. In addition, in some embodiments, the apparatus may include a secondary dispensing module configured to dispense a processing liquid to the container. The processing liquid can be selected from, but not limited to, water, cow milk, soy milk, almond milk, juice, yogurt, soda, lemonade, energy drink, and diary product. In some embodiments, the apparatus can be operatively connected to a processing liquid source (e.g., a water pipe), although, in other embodiments, the apparatus can incorporate a tank or storage for storing one or more processing liquids. In yet more embodiments, the apparatus for making customized nutritional mixtures may include one or more heating modules and/or one or more cooling modules. In yet more embodiments, the apparatus for making customized nutritional mixtures may include a packing module for packing the customized nutritional mixtures in containers (e.g., wrapping, twisting a cap on a bottle, gluing packet edges, etc.). In yet more embodiments, the apparatus for making customized nutritional mixtures may include a labeling module for placing labels on the containers with customized nutritional mixtures. In yet more embodiments, the apparatus for making customized nutritional mixtures may include a transportation module, such as a conveyor.

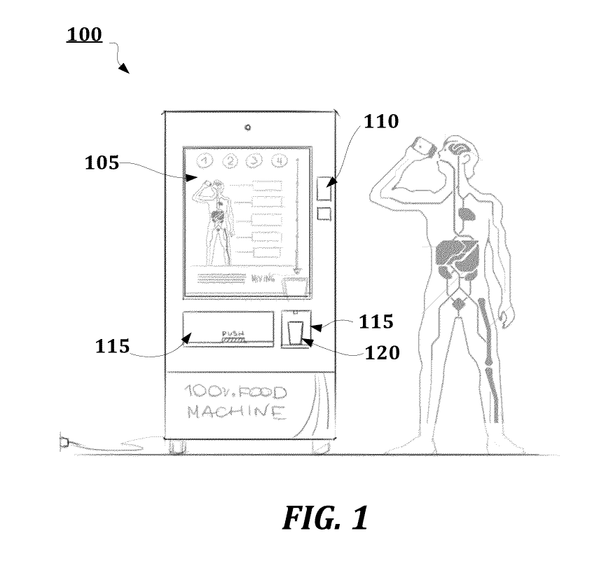

Now, methods and apparatus for making customized nutritional mixtures are described with reference to the drawings. FIG. 1 illustrates a front view of one exemplary apparatus 100 for making customized nutritional mixtures. The apparatus 100 includes a housing within which a number of hardware modules are located. The front panel of the apparatus 100 may include a customer interface such as a touchscreen 105 for displaying information to a customer, as well as for receiving customer's inputs and selections. Notably, instead of the touchscreen 105, there can be utilized a display, such as a Liquid Crystal Display (LCD), and a keypad for acquiring customer inputs. In certain embodiments, there may be no display. In general, the touchscreen 105 can display selections, which can be made by the customer, prompts to make a purchase or utilize the apparatus, or to provide personal or health information. The touchscreen 105 can also display the process of making customized nutritional mixtures, provide nutritional information, guide individuals in making appropriate selections and inputs, and so forth.

In the example shown in FIG. 1, the touchscreen 105 shows a graphical user interface with a number of clickable buttons. The clickable buttons enable the customer to make a desired selection of customized nutritional mixtures. For example, the customer may select among a 2,000 calories diet, low-carb diet, ketogenic diet, high-protein diet, and so forth. The graphical user interface can also include a number of text fields or radio buttons enabling the customer to provide basic biophysical parameters such as an age, gender, height, weight, activity level, although any other biophysical parameters can be also provided.

In additional embodiments, the customer interface can provided on a web site, web service, web resource, server, mobile application, software application installed on a separate computing device, wearable computing device, and so forth.

Still referring to FIG. 1, the apparatus 100 includes an optional payment module 110, which can be configured to accept coins, bank notes, bills, checks, credit, and/or debit cards. In some embodiments, the payment module 110 may enable the individual to make a purchase using a web-interface through monetary or non-monetary instruments including real or virtual currency. The apparatus 100 may also include niches 115 for containers (receptacles) 120 to receive customized nutritional mixture. The apparatus 100 can be operatively connected to a source of electrical power and optionally to a source of processing liquid (e.g., a water source).

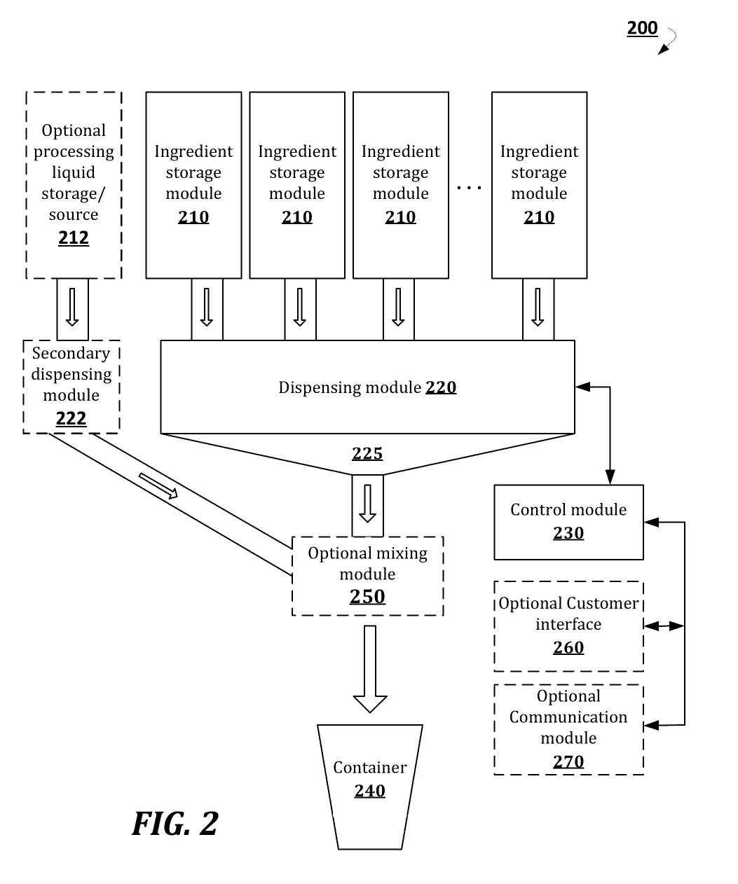

FIG. 2 illustrates a high-level module diagram of exemplary apparatus 200 for making customized nutritional mixtures. As shown in the figure, the apparatus 200 includes one or more ingredient storage modules 210 for storing one or more nutritional ingredients. In some embodiments, one or more of the ingredient storage modules 210 can be used for storing processing liquids.

The nutritional ingredients may refer, but not limited to, bulk ingredients such as dietary fiber, powders, flour, condiments, nutrients, protein, protein isolate, flavor modifiers, texturing ingredients, vitamins, and minerals. Powders may include dairy powders such as whole milk powder, skim milk powder, milk protein concentrate. Powders may also include protein powders such as whey protein powder, soy protein powder, or any other protein derived from a gluten-free plant. The flour ingredients may include grain and other vegetable or fruit flour such as flour of gluten-free plant. The flavor modifying ingredients include condiments such as spices, herbs, salt, sugar, dextrose or other flavor modifying ingredients. Flavors can include artificial flavors or nature flavors such as vanilla, chocolate, and so forth. The nutrients may include seeds of various plants, such as flax seeds, hulled hemp seeds, and the like.

Some ingredient storage modules 210 may contain pre-prepared ingredient mixes to permit faster preparation times. Moreover, the ingredient storage modules 210 can include nutrients or other health-promoting components such as essential fats and fatty acids, saturated fat, cholesterol, vitamins (e.g., vitamin A, vitamin C, vitamin E, vitamin K, vitamin D, vitamin B6, vitamin B12, thiamin, riboflavin, niacin, folate, biotin, and pantothenic acid), minerals (e.g., calcium, iron, phosphorous, iodine, magnesium, zinc, selenium, copper, manganese, chromium, molybdenum, and chloride). Optionally, nutrients may include bioactives such as dried probiotics, bioactive hydrolysate powders and the like.

In some embodiments, the ingredient storage modules 210 can store, but are not limited to flax seed, lecithin granules, hemp hearts, banana bread mix, agar, alginates, arabic, carrageenan, carboxy methyl Cellulose, gelatine, konjac flour, locust bean gum, methyl cellulose and hydroxypropyl methyl cellulose, microcrystalline cellulose, dextrose, pectin, xanthan, acacia gum, bacterial gums, tamarind, ghatti, karaya, galactomannan, gellan polysaccharides, inulin, amylase, amylopectin, exopolysaccharides, maltodextrin, gelatine, fibre, protein, transglutaminase or hydrolysate products, amino acids, antioxidants, sugars, ginseng, guarana, and/or caffeine.

Notably, each of the ingredient storage modules 210 may contain only one of the listed above ingredients. However, in certain embodiments, some of the ingredient storage modules 210 may include a mixture of the listed above ingredients.

In some embodiments, some of ingredient storage modules 210, such as optional processing liquid storage 212, can contain processing (consumable) liquids such as water, milk, diary products (e.g., yogurt), juice, non-diary milk (e.g., soymilk or almond milk), lemonades, energy drinks, coffee, tea, or other potable liquid. However, in alternative embodiments, processing liquids can be delivered from a remote liquid source, e.g. a water source. For these ends, the apparatus 200 may be operatively coupled to, for example, a water pipe.

Moreover, in some embodiments, refrigerator or cooling means can be provided for some ingredient storage modules 210 where the ingredients require such conditions. Where ingredients need to be kept at elevated temperatures, heating devices can be also included.

Still referring to FIG. 2, the apparatus 200 further includes a dispensing module 220 for selective dispensing nutritional ingredients to a container. Accordingly, the dispensing module 220 can be connected to the ingredient storage modules 210 through one or more conduits, tubes, and/or buckets. The dispensing module 220 can refer to a linear weigh filler, which can include multiple batching units such as shutters, valves, control gates, sealers, trims, or the like devices, each of which is operatively coupled to ingredient storage modules 210. The batching units are selectively controlled by a control module 230 such that only a number of specific nutritional ingredients can be dispensed in a particular dosage. In some embodiments, there can be provided an additional, but optional, secondary dispensing module 222 for delivering processing, consumable liquids from the storage 222 to a container 240.

The dispensing module 220 can also include a hopper 225 for directing dispensed nutritional ingredients to a container 240 or optional mixing module 250. The nutritional ingredients are advanced to the container 240 and/or optional mixing module 250 by means of gravity, although augers, conveyor line, air or liquid pressure can be employed as well. The mixing module 250 can include one or more mixers for mixing dispensed nutritional ingredients. In some embodiments, there can be provided one or more processing liquids into the container 240 and/or optional mixing module 250. If the mixing module 250 is utilized, mixed product is delivered to the container 240 through a conduit from the mixing module 250. In other embodiments, mixing or stirring can occur inside the container 240. The mixing module 250 can also heat or chill the mixture of nutritional ingredients (if required). In yet more embodiments, one or more processing modules can also be used for shaking, backing, and/or cooking of the nutritional mixtures. In yet more embodiments, one or more post-processing modules can be also used for packaging in containers 240, labeling containers 240, and transporting containers 240. In yet more embodiments, the apparatus 200 optionally includes a storage of containers and a release module for releasing containers to one of the niches 115 below the dispensing module 220 or optional mixing module 250.

Still referring to FIG. 2, the apparatus 200 may further include a customer interface 260 for displaying information to the customer and receiving inputs of the customer. As discussed above, the customer interface 260 can include, but not limited to, a touchscreen, display, keypad, keyboard, trackball, speaker, or any other suitable input and/or output device. In other words, the customer interface 260 may include one or more input devices and/or one or more output devices depending on particular needs.

In yet more embodiments, the apparatus 200 may further include a communications module 270 such as a modem, network interface, a wireless transceiver, and so forth. The communications module 270 is configured to operatively communicate with remote devices over a communications network such as the Internet, intranet, cellular network, LAN, WAN, IEEE 802.11 based network, and so forth. In certain embodiments, communications module 270 is configured to receive requests to make customized nutritional mixtures, individual preferences, instructions, selections, biophysical parameters, etc. from a remote server, website, web service, web resource, mobile device, remotely located computing device, and/or wearable computer. For example, a request to make customized nutritional mixtures can be created by an individual on his mobile device or via website, which transfer it to the apparatus 200, while at least some biophysical parameters are obtained from a wearable computing device (e.g., a wearable physical activity tracker) and/or user profile stored on a remote web resource. It shall be understood that information required to make customized nutritional mixtures can be obtained from one remote device (e.g., a server or mobile device) or multiple remote devices (e.g., some information is provided from a mobile device, some information is provided from a wearable computing device, some information is provided from a web service). The communications module 270 can also send status updates, alerts and notifications to one or more remote devices such as servers, mobile devices, and wearable computing devices.

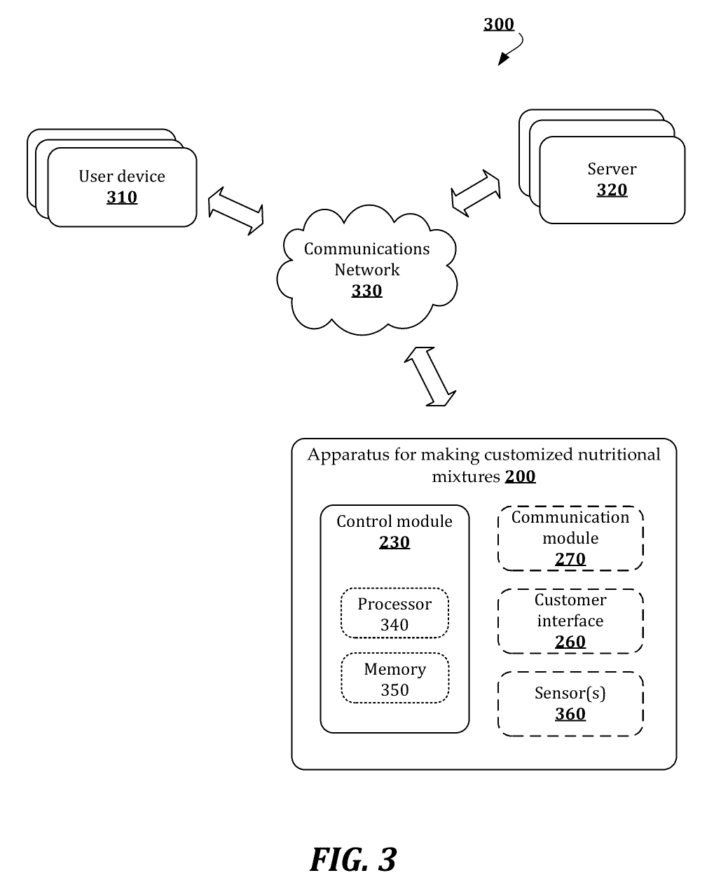

These principles are further illustrated in FIG. 3, which shows a system environment 300 for operating an apparatus 200 for making customized nutritional mixtures. As shown in this figure, the apparatus 200 can operatively communicate with one or more user devices 310 and/or one or more servers 320 via one or more communications networks 330.

The term “user device,” as used herein, shall mean any suitable computing device operable by a user such as an individual who wants to consume a customized nutritional mixture. Examples of user devices 310 include a computer (e.g., a desktop computer, laptop computer, and tablet computer), mobile phone, smart phone, and wearable computing device (e.g., a sport activity tracker (watch, bracelet) with one or more sensors for sensing vital or biophysical parameters).

Servers 320 may run a website, web service or web resource that can store user profiles including various characteristics, parameters or preferences of individuals. For example, one of the servers 320 may run an internet store for ordering customized nutritional mixtures, where the internet store allows individuals to generate requests to prepare customized nutritional mixtures based on their inputs. The internet store can accept payments, track orders, send notifications, and so forth.

The communication network 330 can include one or more wired, wireless, or optical networks including, for example, the Internet, intranet, PAN (Personal Area Network), LAN (Local Area Network), WAN (Wide Area Network), MAN (Metropolitan Area Network), virtual private network (VPN), storage area network (SAN), Ethernet network, ISDN (Integrated Services Digital Network), GSM (Global System for Mobile Communication) network, CDMA (Code Division Multiple Access) network, TDMA (Time Division Multiple Access) network or any other cellular phone networks, Bluetooth radio, an IEEE 802.11-based radio frequency network, among others.

As further illustrated in FIG. 3, the control module 230 may refer to a computing device and include one or more processors 340 and a memory 350. In various embodiments, the control module 230 refer, but not limited to a personal computer (e.g., a desktop computer, laptop computer, tablet computer) utilizing a central control processing system, which utilizes a central processing unit (CPU), memory, and an interconnect bus. The one or more processors 340 may contain a single microprocessor, CPU, or it may contain a plurality of microprocessors for configuring the one or more processors 340 as a multi-processor system. The memory 350 includes a main memory, such as a dynamic random access memory (DRAM) and cache, as well as a read-only memory, such as a PROM, EPROM, FLASH-EPROM, or the like. In operation, the memory stores at least portions of processor-executable instructions for execution by the one or more processors 340 and data for processing in accord with the executed instructions.

Although summarized above as a PC-type implementation, those skilled in the art will recognize that the control module 230 also encompass systems such as host computers, servers, workstations, network terminals, and the like. Hence, aspects of the systems and methods provided herein encompass hardware and software for controlling the relevant functions. Software may take the form of code or executable instructions for causing the control module 230 or other programmable equipment or logic to perform the relevant steps, where the code or instructions are carried by or otherwise embodied in a medium readable by the processor or another machine. Instructions or code for implementing such ope ratio may be in the form of computer instruction in any form (e.g., source code, object code, interpreted code, etc.) stored in or carried by any tangible readable medium.

As used herein, terms such as computer or machine “readable medium” refer to any medium that participates in providing instructions to a processor for execution. Such a medium may take many forms. Non-volatile storage media include, for example, optical or magnetic disks, such as any of the storage devices in any computer(s) shown in the drawings. Volatile storage media include dynamic memory. Common forms of computer-readable media therefore include, for example: hard disk, magnetic tape, any other magnetic medium, a CD-ROM, DVD, any other optical medium, RAM, PROM and EPROM, FLASH-EPROM, any other memory chip or cartridge, or any other medium from which the processor can read programming code and/or data. Many of these forms of computer readable media may be involved in carrying one or more sequences of one or more instructions to a processor for execution.

In certain embodiments, there can be provided one or more sensors 360 to measure weight, temperature, dosage or amount of nutritional ingredients, which remain in the storage modules 210. Moreover, in yet other embodiments, the apparatus 200 may include one or more sensors 360 (e.g., digital video cameras, biosensors, or measurement devices) for acquiring customer biophysical or vital parameters including, but not limited to, a individual height, individual weight, individual dimensions, various vital parameters associated with the customer such as blood pressure, heart rate, blood oxygen level, bone thickness, and so forth.

Referring back to FIG. 2, the control module 230 is operatively coupled at least to the dispensing module 220, optional customer interface 260 (if used), optional communication module 270 (if used), and optional mixing module 250 (if used). Accordingly, the operation of the apparatus 200 can be fully automated although, in some embodiments, certain operations may include actions of the individual or an operator. For example, the individual may need to place a container 240 (e.g., a cup or bottle) under the dispensing module 220 and push a start button to initiate the operation of the apparatus 200. The control module 230 also provides interactive interaction with the customer, customer's mobile device and/or remote resources.

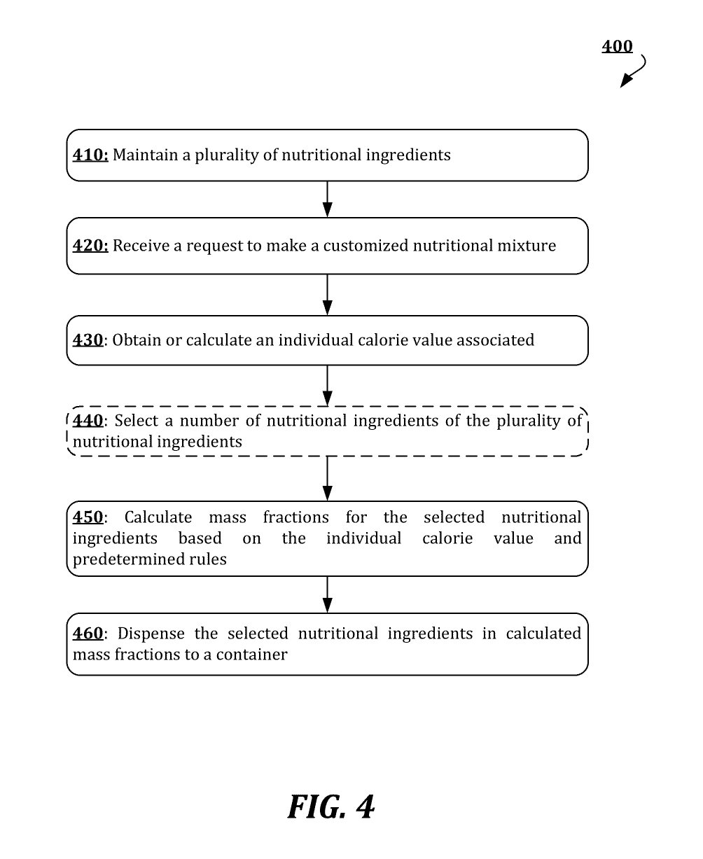

FIG. 4 is a process flow diagram showing a method 400 for making customized nutritional mixtures, according to an example embodiment. The method may be performed by processing logic that may comprise hardware (e.g., decision-making logic, dedicated logic, programmable logic, and microcode), software (such as software run on a general-purpose computer system or a dedicated machine), or a combination of both. In one example embodiment, the processing logic refers to one or more components of apparatus 220 and optionally system environment 300. Notably, the below-recited steps of method 400 may be implemented in an order different than described and shown in FIG. 4. Moreover, method 400 may have additional steps not shown herein, but which can be evident for those skilled in the art from the present disclosure. Method 400 may also have fewer steps than outlined below and shown in FIG. 4.

Method 400 commences at step 410 with maintaining a plurality of nutritional ingredients in one or more ingredient storage modules 210. As discussed above, the nutritional ingredients may include bulk ingredients such as dietary fiber, powders, flour, condiments, nutrients, protein, protein isolate, flavor modifiers, texturing ingredients, vitamins, and minerals. Some ingredients may include consumable liquids such as water, milk, diary products, soda, lemonade, energy drink, coffee, tea, soymilk, almond milk, and so forth.

At step 420, the control module 230 receives a request to make a customized nutritional mixture via a communication module 270 and/or customer interface 260. The request can be generated when an individual interacts with the customer interface 260, user device 310 (through a software application), and/or website or web service on server 320. Each request to make a customized nutritional mixture is associated with a particular individual or a group of individuals.

In certain embodiments, each request to make a customized nutritional mixture includes one or more biophysical parameters of the individual. The biophysical parameters may include a gender of the individual, an age of the individual, a height of the individual, a weight of the individual, and a daily activity level of the individual (e.g., sedentary, light, medium or heavy). In certain embodiments, each request to make a customized nutritional mixture further includes individual goals such as to maintain weight and eat healthy, lose weight, or gain weight or muscles. For these ends, the individual can provide answers to multiple questions or make appropriate selections through the customer interface 260 or user device 310.

In yet more embodiments, each request to make a customized nutritional mixture may include a serving selection, flavor selection, amount and/or weight of customized nutritional mixture, one or more customer preferences, a type of container to be utilized for receiving the customized nutritional mixture, and so forth.

In yet more embodiments, each request to make a customized nutritional mixture may further include one or more of the following vital parameters: a blood pressure of the individual, a heart rate of the individual, a blood oxygen level of the individual, a value of calories consumed by the individual within a predetermined period, and a bone thickness of the individual. In yet more embodiments, each request to make a customized nutritional mixture may further include one or more of the following physical activity parameters: a number of steps made by the individual for a period of time, a distance travelled by the individual, sleep quality parameters of the individual, a number of calories burned off by the individual, and a type of physical activity of the individual

In certain embodiments, one or more of the biophysical parameters, vital parameters, and/or physical activity parameters can be automatically measured by one of the sensors 360 and/or by sensors employed by one or more of the user devices 310. In other embodiments, one or more of the biophysical parameters, vital parameters, and/or physical activity parameters can be manually input by the individual or an operator. In other embodiments, one or more of the biophysical parameters, vital parameters, and/or physical activity parameters can be retrieved from a database, website, web resource, and user profile.

At step 430, the control module 230 obtains an individual calorie value associated with the individual. The individual calorie value may be obtained differently. In one example, the individual calorie value is manually input by the individual using the customer interface 260, user device 310 or server 320. In another example, the individual calorie value is received from a database, user profile, user device 310, remote server 320, website, web resource, or web service. In some embodiments, the individual calorie value can be included in the request of the individual to make the customized nutritional mixture. In this case, the obtaining of the individual calorie value includes retrieving the individual calorie value from the request.

In yet another example, the individual calorie value is calculated by the control module 230 based on information given in the request to make a customized nutritional mixture and/or one or more of the biophysical parameters, vital parameters, and/or physical activity parameters, which are listed above.

One of the important aspect of the present technology is that the individual calorie value as received or calculated by the control module 230 characterizes a recommended target value of calories to be burned off by the individual within a predetermined period, where the predetermined period is between 1 hour and 24 hours. In other words, the term “individual calorie value” means, in certain embodiments, a calorie value recommended for an individual to intake within a predetermined period. The predetermined period may include 24 hours. Thus the “individual calorie value” means individual daily calorie value. In other embodiments, the predetermined period may be less than 24 hours, and it can be, for example, 1 hour, 2 hours, 3 hours, and so forth. Thus, the individual calorie value can be calculated regardless on a period when that amount of calories is to be consumed. Accordingly, the individual calorie value may be within the range of 100 calories and 10,000 calories, and more preferably within the range 100 calories and 4,000 calories. Some typical individual daily calorie values are 1,800 calories, 2,000 calories, 2,200 calories, 2,400 calories, 2,600 calories, 2,800 calories, 3,000 calories, and so forth. In some embodiments, the “individual calorie value” can be calculated on a 12-hour basis, and in this case, typical individual calorie values are 900 calories, 1,000 calories, 1,100 calories, 1,200 calories, 1,300 calories, and so forth. In yet more embodiments, the present technology allows for calculating the “individual calorie value” for each meal. In the latter case, typical individual calorie values can be 100 calories, 150 calories, 200 calories, 250 calories, 300 calories, 350 calories, 400 calories, and so forth. A standard deviation of the above-given values can be within 20%, more preferably within 10%.

Generally, the recommended target calorie value may vary from about 1,000 calories per day to about 4,000 calories per day depending on individual characteristics and goals. However, it shall be understood the calorie value can be less than 1,000 calories per day (e.g., for children) or greater than 4,000 calories per day depending on individual characteristics of the individual. In one example, the recommended target calorie value is 2,000 calories per day. In another example, the recommended target calorie value is between 1,900 and 2,100 calories per day. In another example, the recommended target calorie value is between 2,000 and 2,300 calories per day. In another example, the recommended target calorie value is between 1,700 and 2,000 calories per day. It shall be clear that the recommended target calorie value can be of various ranges.

The individual calorie value can be calculated by the control module 230 based upon Body Mass Index (BMI), Basal Metabolic Rate (BMR) and/or Resting Metabolic Rate (RMR) associated with the individual. In other embodiments, the calorie value can be calculated based on Mifflin-St Jeor equation, Katch-McArdle equation, Cunningham equation, Harris-Benedict equation, and any combination thereof.

At optional step 440, the control module 230 selects a number of nutritional ingredients of the plurality of nutritional ingredients stored in the storages 210 based on the predetermined nutritional rules and individual preferences. The predetermined nutritional rules, in general, enable the control module 230 to make selection of certain nutritional ingredients and their dosages such that, when the selected ingredients are mixed, the mixed product provides complete nutritional elements as recommended by nutritional standards (e.g., USDA standards). For example, if it is calculated that a particular customer needs to digest about 1,900-2,100 calories per day, the predetermined nutritional rules enable the apparatus 200 to create a customized nutritional mixture, which would include all required nutrients, vitamins, minerals, fiber and/or other components in daily values, which include 1,900-2,100 calories and which required to digest by an individual as prescribed, for instance, by the U.S. Food and Drug Administration (USFDA) regulations, USDA regulations, or any other government or non-government recommendations. Accordingly, when the customized nutritional mixture consumed by the individual, he obtains all necessary nutrition for entire day in the dosages and consistency that is optimal for his age, height, weight, activity habits, goals, and so forth.

Notably, the selection of nutritional ingredients depends on the calorie value and optionally other customer parameters discussed above. It shall be understood that one customer would get one combination of nutritional ingredients while another customer would get another combination of nutritional ingredients. In some embodiments, however, the step 3 is optional or not required.

One of the important aspects of this technology is that the predetermined rules include one or more ratios of the nutritional ingredients associated with multiple calorie values. In other words, the predetermined rules can provide mapping of the calorie value obtained to one or more ratios of the nutritional ingredients.

The ratio of the nutritional ingredients may be divided into first ratios and second ratios. The first ratios are associated with calorie sources such as fats, carbohydrates, and proteins. The second ratios associated with minerals and vitamins. In general, the vitamins include at least: Vitamin A, Vitamin B6, Vitamin B12, Vitamin C, Vitamin D, Vitamin E, Vitamin K, Thiamin, riboflavin, niacin, folate, and choline. Minerals include at least: calcium, copper, iron, magnesium, phosphorous, selenium, zinc, potassium, and sodium. In one example, the first ratios include 10%-40% of fats, 40%-80% of carbohydrates and 10%-50% of proteins. In another example, the first ratios include about 30% of fats, about 60% of carbohydrates and about 10% of proteins.

Accordingly, the nutritional ingredients can be selected such that the selected nutritional ingredients correspond to at least one of the first ratios and at least one of the second ratio. The selection is based on the biophysical parameters of the customer and the individual calorie value. For example, the control module 230 can select one of the ratio of the nutritional ingredients based on a gender of the individual, an age of the individual, a height of the individual, a weight of the individual, and a daily activity level of the individual.

At step 450, the control module 230 calculates mass fractions (dosages) for the selected nutritional ingredients based on the individual calorie value and predetermined rules. Similar to step 440, the mass fractions (dosages) depend on the individual calorie value and optionally other parameters listed above. Those skilled in the art will understand that one individual would get nutritional ingredients mixed with mass fractions according to one rule while another customer would get nutritional ingredients mixed with mass fractions according to another rule.

The predetermined rules used to calculate the mass fractions include one or more nutritional ingredient selection rules associated with a particular calorie diet. The calorie diet is associated with the individual and based on 1,000 to 4,000 calorie daily intake. In other words, the mass fractions depend on the ratio of the nutritional ingredients as discussed above. In some embodiments, the control module 230 maps one or more ratios of the nutritional ingredients to the individual calorie value associated the individual.

Once a particular ratio is determined for a particular individual based on his individual calorie value and/or biophysical parameters, the control module 230 can retrieve or calculate corresponding mass fractions of select nutritional ingredients. In one embodiment, optimal amounts of nutritional ingredients (i.e., daily values) can have the following limitations (for the 2,000 calorie diet):
Total Fat: about 65 grams;
Saturated Fat: about 20 grams;
Cholesterol: about 300 milligrams;
Sodium: about 2,400 milligrams;
Potassium: about 3,500 milligrams;
Total Carbohydrate: about 300 grams;
Dietary Fiber: about 25 grams;
Protein: about 50 grams;
Vitamin A: about 5,000 IU;
Vitamin C: about 60 milligrams;
Calcium: about 1,000 milligrams;
Iron: about 18 milligrams;
Vitamin D: about 400 IU;
Vitamin E: about 30 IU;
Vitamin K: about 80 micrograms;
Thiamin: about 1.5 milligrams;
Riboflavin: about 1.7 milligrams;
Niacin: about 20 milligrams;
Vitamin B6: about 2 milligrams;
Folate: about 400 micrograms;
Vitamin B12: 6 micrograms;
Biotin: about 300 micrograms;
Pantothenic Acid: about 10 milligrams;
Phosphorus: about 1,000 milligrams;
Iodine: about 150 micrograms;
Magnesium: about 400 milligrams;
Zinc: about 15 milligrams;
Selenium: about 70 micrograms;
Copper: about 2 milligrams;
Manganese: about 2 milligrams;
Chromium: about 120 micrograms;
Molybdenum: about 75 micrograms; and
Chloride: about 3,400 milligrams.

In some embodiments, the above-listed nutrients can be supplemented with additional nutrients. In other embodiments, one or more nutrients can be excluded from the list. In any case, it shall be understood that the above-given list of nutrients is an example list.

At step 460, the control module 230 controls the dispensing module 220 and causes it to dispense the selected nutritional ingredients in calculated mass fractions to the container 240 and/or mixing module 250.

In yet more embodiments, the control module 230 can optionally dispense a liquid to the container by controlling an optional secondary dispensing module shown in FIG. 2.

If mixing is required, the control module 230 also controls the mixing module 250 such that the selected nutritional ingredients are mixed or stirred to a preferable consistency. When mixed, the mixed nutritional ingredients are delivered to the container 240. Alternatively, the mixing can occur inside the container 240.

Importantly, a calorie value of the customized nutritional mixture as delivered to the container 240 substantially corresponds to at least a predetermined portion of the individual calorie value. In one example, the calorie value of the customized nutritional mixture is substantially equal to the individual calorie value, which was used as a target value. In other words, the calorie value of prepared customized nutritional mixture can correspond to an individual daily calorie value (which can be 2,000 calories in one example embodiment). The term “substantially” means that a standard deviation in actual calorie value of the customized nutritional mixture may vary within 20 percent, and more preferably within 10 percent (e.g., if an individual daily calorie value is 2,000, the actual calorie value of the customized nutritional mixture can be within the range of 1,900-2,100 range). In another example, the calorie value of the customized nutritional mixture is more than the individual calorie value.

In another example, the calorie value of the customized nutritional mixture as provided in a container 240 is substantially a portion of the individual calorie value, which was used as a target value. For instance, this portion can be between 10% and 50% of the individual calorie value. In other words, he the apparatus 200 can provide one or more containers 240 with a customized nutritional mixture, where each of containers 240 may have as low as 10% of the individual calorie value such as the individual daily calorie value. Dividing customized nutritional mixture into portions may be necessary to create several daily meals or individual servings such as breakfast, lunch, and dinner. Each of the meals or servings can be provided in a separate container 240. Each portion can have equal or varying calorie values. For example, the method 400 may produce three containers 240 with customized nutritional mixture, where each of the containers 240 includes one-third of the individual calorie value (e.g., if the individual daily calorie value is 2,000 calories, then each portion will include a customized nutritional mixture having about 667 calories). In another example, however, the method 400 may produce three containers 240 with customized nutritional mixture, where first container includes 50% of the individual calorie value, second container includes 30% of the individual calorie value, and third container includes 20% of the individual calorie value. It shall be clear that other proportions and other number of containers can be used. For example, customized nutritional mixture can be created for an individual each time he wants to eat. In this case, the apparatus 200 will obtain his individual daily calorie value, determine how many calories he has already consumed, and make a customized nutritional mixture to be consumed as another meal.

In yet more embodiments, the method 400 can be practiced to produce more than individual servings of customized nutritional mixture. For example, nutritional ingredients can be dispensed in mass fractions to constitute multiple servings of predetermined calorie value. Multiple servings can be dispensed into a single container so that all servings in aggregate can be considered as a bulk nutritional mixture. Individuals are then needed to take appropriate amounts of the mixture from the container for further consuming. Generally, aggregate calorie value of customized nutritional mixture prepared in this manner may be more than 4,000 calories.

The method 400 may be practiced to make customized nutritional mixture including various ingredients and using various recipes. In one example recipe, there are used flax seeds and hulled hemp seeds as main ingredients, which can be supplemented with additional ingredients. In another example recipe, there are used: flax seeds, hulled hemp seeds, flour from at least one gluten-free plant, dextrose, protein isolate derived from at least one gluten-free plant, and protein derived from at least one gluten-free plant. In these example recipes, total fat is about 30%, carbs are between about 40-60%, and protein is between about 10-30% depending on individual goals (e.g., to remain weight, to lose weight, to gain weight).

For these recipes, the following mass fractions can be selected (in grams per 100 calories):
Flax seed: about 4.5-5.2
Hulled hemp seed: about 1.7-2.7
Rice flour: about 2.7-8.0
Dextrose: 1.1-3.7
Optional soy protein: about 0-3.8
Optional soy protein isolate: about 1.1-5.8
Optional hulled sesame seeds: about 0.3-0.8
Optional lecithin: about 2.4

It was shown that the above mass fractions of the selected ingredients provide about 97-98% of complete nutrients, vitamins, and minerals required according to USDA 2,000 calorie diet.

Thus, the methods and apparatus for making customized nutritional mixtures have been described. Although embodiments have been described with reference to specific example embodiments, it will be evident that various modifications and changes can be made to these example embodiments without departing from the broader spirit and scope of the present application. Accordingly, the specification and drawings are to be regarded in an illustrative rather than a restrictive sense.

What is claimed is:

1. A method for making customized nutritional mixtures, the method comprising:
maintaining a plurality of nutritional ingredients;
receiving, by means of a control module comprising a processor and a memory storing program instructions executable by the processor, a request to make a customized nutritional mixture, wherein the request is associated with an individual;
obtaining, by means of the control module, an individual calorie value associated with the individual;
calculating, by means of the control module, mass fractions of nutritional ingredients based on the individual calorie value; and
dispensing, by means of a dispensing module, selected nutritional ingredients in accordance with the mass fractions to a container in order to produce the customized nutritional mixture, wherein a calorie value of the customized nutritional mixture substantially corresponds to a predetermined portion of the individual calorie value.

2. The method of claim 1, further comprising:
selecting, by means of the control module, a number of nutritional ingredients of the plurality of nutritional ingredients based on one or more predetermined rules and individual preferences.

3. The method of claim 1, further comprising:
dispensing, by means of a secondary dispensing module, a consumable liquid to the container.

4. The method of claim 1, further comprising:
mixing, by means of a mixing module, the selected nutritional ingredients inside the container.

5. The method of claim 1, wherein the individual calorie value characterizes a recommended calorie target value for the individual to consume within a predetermined period, wherein the predetermined period is between 1 hour and 24 hours.

6. The method of claim 1, wherein the request of the individual to make the customized nutritional mixture is received from a customer interface based on an input of the individual.

7. The method of claim 1, wherein the individual calorie value is included in the request of the individual to make the customized nutritional mixture, and the obtaining of the individual calorie value includes retrieving the individual calorie value from the request.

8. The method of claim 1, wherein the obtaining of the individual calorie value comprising receiving, by means of the control module, the individual calorie value calorie value from a mobile device associated with the individual.

9. The method of claim 1, wherein the obtaining of the individual calorie value comprising receiving, by means of the control module, the individual calorie value from a wearable computing device associated with the individual, wherein the wearable computing device includes at least one sensor for sensing one or more vital parameters of the individual.

10. The method of claim 1, wherein the obtaining of the individual calorie value comprising receiving, by means of the control module, the individual calorie value from a website, a web service, and/or a web resource.

11. The method of claim 1, further comprising:
receiving, by means of the control module, biophysical parameters of the individual;
wherein the obtaining of the individual calorie value comprising calculating the individual calorie value based upon the biophysical parameters of the individual.

12. The method of claim 11, wherein the biophysical parameters of the individual comprising a gender of the individual, an age of the individual, a height of the individual, a weight of the individual, and a daily activity level of the individual.

13. The method of claim 11, wherein the biophysical parameters of the individual comprising one or more of the following vital parameters: a blood pressure of the individual, a heart rate of the individual, a blood oxygen level of the individual, a value of calories consumed by the individual within a predetermined period, and a bone thickness of the individual.

14. The method of claim 11, wherein the biophysical parameters of the individual comprising one or more of the following physical activity parameters: a number of steps made by the individual for a period of time, a distance travelled by the individual, sleep quality parameters of the individual, a number of calories burned off by the individual, and a type of physical activity of the individual.

15. The method of claim 11, wherein the calculating of the calorie value is based on a Body Mass Index (BMI) associated with the individual.

16. The method of claim 11, wherein the calculating of the calorie value is based on a Basal Metabolic Rate (BMR) or a Resting Metabolic Rate (RMR) associated with the individual.

17. The method of claim 1, wherein the calculating of the mass fractions of the nutritional ingredients is further based on one or more nutritional ingredient selection rules associated with a calorie diet, wherein the calorie diet is associated with the individual and based on 1,000 to 4,000 calorie daily intake.

18. The method of claim 17, wherein the predetermined rules include one or more ratios of the nutritional ingredients associated with multiple calorie values.

19. The method of claim 18, further comprising:
mapping, by means of the control module, the one or more ratios of the nutritional ingredients to the individual calorie value associated the individual.

20. The method of claim 18, wherein the one or more ratios of the nutritional ingredients comprising first ratios associated with calorie sources and second ratios associated with minerals and vitamins, wherein the calorie sources include fats, carbohydrates, and proteins.

21. The method of claim 20, wherein the vitamins comprising: Vitamin A, Vitamin B6, Vitamin B12, Vitamin C, Vitamin D, Vitamin E, Vitamin K, Thiamin, riboflavin, niacin, folate, and choline; and wherein the minerals comprising calcium, copper, iron, magnesium, phosphorous, selenium, zinc, potassium, and sodium.

22. The method of claim 20, wherein the calculating of the mass fractions of the selected nutritional ingredients is performed such that the selected nutritional ingredients correspond to at least one of the first ratios and at least one of the second ratio.

23. The method of claim 20, wherein the first ratios include: 10%-40% of fats. 40%-80% of carbohydrates, and 10%-50% of proteins.

24. The method of claim 20, wherein the first ratios include: about 30% of fats, about 60% of carbohydrates, and about 10% of proteins.

25. The method of claim 20, further comprising:
selecting, by means of the control module, one of the ratio of the nutritional ingredients based on a gender of the individual, an age of the individual, a height of the individual, a weight of the individual, and a daily activity level of the individual.

26. The method of claim 20, further comprising:
selecting, by means of the control module, one of the ratio of the nutritional ingredients based on one or more of the biophysical parameters associated with the individual.

27. The method of claim 1, wherein the nutritional ingredients include flax seeds and hulled hemp seeds.

28. The method of claim 1, wherein the nutritional ingredients include: flax seeds, hulled hemp seeds, flour from at least one gluten-free plant, dextrose, and protein.

29. The method of claim 1, wherein the predetermined portion of the individual calorie value is between 10% and 50% of the individual calorie value, wherein the individual daily calorie value is between 100 calories and 4,000 calories.

30. The method of claim 1, wherein the predetermined portion of the individual daily calorie value is at least 100% of the individual daily calorie value, wherein the individual daily calorie value is between 100 calories and 4,000 calories.

31. An apparatus for making customized nutritional mixtures, the apparatus comprising:
a plurality of ingredient storage modules for storing a plurality of nutritional ingredients;
a dispensing module for dispensing nutritional ingredients to a container;
a control module comprising a processor and a memory storing program instructions executable by the processor, the control module is operatively coupled to the dispensing module, wherein the control module is configured to:
receive a request to make a customized nutritional mixture, wherein the request is associated with an individual;
obtain an individual calorie value associated with an individual;
calculate mass fractions of nutritional ingredients based on the individual calorie value; and
cause the dispensing module to dispense selected nutritional ingredients in accordance with the mass fractions to a container in order to produce the customized nutritional mixture, wherein a calorie value of the customized nutritional mixture substantially corresponds to at least a predetermined portion of the individual calorie value.

32. The apparatus of claim 31, further comprising:
a customer interface for displaying information to the individual and for receiving the request of the individual.

33. The apparatus of claim 31, further comprising:
a communication module for receiving the request of the individual from a mobile device, a wearable computer, or a server.

34. A non-transitory processor-readable medium having instructions stored thereon, which when executed by one or more processors, cause the one or more processors to implement a method for making customized nutritional mixtures, the method comprising:
receiving a request to make a customized nutritional mixture, wherein the request is associated with an individual;
obtaining an individual calorie value associated with an individual;
calculating mass fractions of nutritional ingredients based on the individual calorie value; and
causing to dispense selected nutritional ingredients in accordance with the mass fractions to a container in order to produce the customized nutritional mixture, wherein a calorie value of the customized nutritional mixture substantially corresponds to at least a predetermined portion of the individual calorie value.
